(19) United States
(12) Patent Application Publication
(10) Pub. No.: US 2023/0107402 A1
(43) Pub. Date: Apr. 6, 2023

(54) SYSTEMS, DEVICES, AND METHODS FOR MONITORING INDOOR AIR

(71) Applicant: Good Robot Monitoring Inc., Toronto (CA)

(72) Inventors: Alan Majer, Toronto (CA); Mark Edward Loit, Toronto (CA)

(21) Appl. No.: 17/527,810

(22) Filed: Nov. 16, 2021

Related U.S. Application Data
(60) Provisional application No. 63/262,005, filed on Oct. 1, 2021.

(51) Int. Cl. F24F 11/63; F24F 11/52

(52) U.S. Cl. CPC F24F 11/63; F24F 11/52; F24F 2110/64

(57) ABSTRACT

Systems, devices, and methods are provided for monitoring air in an indoor space. An example system includes at least one sensor located in the indoor space to generate monitoring data, a communication interface, and a processor connected to the communication interface and in communication with the at least one sensor. The at least one sensor includes at least a carbon dioxide sensor or a particle sensor. The processor is operable to receive monitoring data for an extended time period, which includes at least one period when the indoor space is occupied and at least one period when the indoor space is unoccupied. The processor is operable to identify at least one region of interest in the monitoring data; and determine an air change rate for the indoor space based on the region of interest. The processor can be further operable to determine an occupancy or an airborne infection risk based on real-time monitoring data.

### CROSS-REFERENCE TO RELATED PATENT APPLICATIONS

This application claims priority from U.S. Provisional Patent Application No. 63/262,005, entitled “Systems, Devices, and Methods for Monitoring Indoor Air”, filed on Oct. 1, 2021. The entire contents of U.S. Provisional Patent Application No. 63/262,005 is herein incorporated by reference for all purposes.

### FIELD

The described embodiments relate to systems, devices, and methods for monitoring indoor air. In some example embodiments, the systems, devices, and methods can relate to determining occupancy in an indoor space. In some example embodiments, the systems, devices, and methods can relate to determining airborne infection risk in an indoor space.

### BACKGROUND

Indoor air can contain various pollutants such as carbon monoxide (CO), carbon dioxide (CO2), nitrogen dioxide (NO₂), radon (Rn), volatile organic compounds (VOCs), and biological contaminants, amongst other things. Biological contaminants can include but is not limited to bacteria, viruses, pollens, dust, and mold. Indoor air pollutants can occur naturally or from sources such as fossil fuel-burning appliances, building materials and furnishings, household products, heating, ventilation, and air conditioning (HVAC) devices, excess moisture, and human activities such as breathing, talking, or smoking.

Indoor air quality can affect the health and comfort of occupants. Immediate effects can include transmission of airborne diseases and aggravation of asthma and other symptoms, similar to colds or viral diseases. Long term effects can include respiratory diseases, heart disease, and cancer. Indoor air quality can also impact cognitive function. Indoor air can be monitored to understand and mitigate the risks associated with poor indoor air quality.

### SUMMARY

The various embodiments described herein generally relate to methods (and associated systems and devices configured to implement the methods) for monitoring indoor air. The disclosed systems and methods can relate to determining occupancy in an indoor space. The disclosed systems and methods can relate to determining airborne infection risk in an indoor space.

An example system can include at least one sensor located in the indoor space to generate monitoring data, a communication interface, and at least one processor connected to the communication interface and in communication with the at least one sensor. The at least one sensor can include at least one of a carbon dioxide sensor to detect carbon dioxide concentration within the indoor space or a particle sensor to detect airborne particles within the indoor space. The at least one processor can be operable to receive, from the at least one sensor, monitoring data for an extended time period, identify at least one region of interest in the monitoring data; and determine an air change rate for the indoor space based on the at least one region of interest. The extended time period can include at least one period when the indoor space is occupied and at least one period when the indoor space is unoccupied.

In at least one embodiment, each region of interest identified in the monitoring data can include a portion of the monitoring data having a substantially constant rate of decay immediately followed by a portion of the monitoring data being substantially constant.

In at least one embodiment, for each region of interest, the monitoring data at the start of the region of interest can be greater than a pre-determined minimum value.

In at least one embodiment, for each region of interest, the monitoring data at the end of the region of interest can be greater than a pre-determined maximum value.

In at least one embodiment, the at least one sensor further can include a barometric pressure sensor to detect pressure changes within the indoor space; and the at least one processor can be operable to: identify transitional ventilation events in the monitoring data based on the pressure changes detected by the barometric pressure sensor; and identify the at least one region of interest in the monitoring data based on the transitional ventilation events.

In at least one embodiment, the at least one processor can be further operable receive, from the at least one sensor, real-time monitoring data; determine at least one of an occupancy in the indoor space based on the real-time monitoring data; or an airborne infection risk in the indoor space based on the real-time monitoring data; and cause, via the communication interface, the occupancy or the airborne infection risk to be displayed.

In at least one embodiment, the at least one processor can be operable to: determine a safe occupancy capacity based on the air change rate; and determine the airborne infection risk based on the safe occupancy capacity and the real-time monitoring data.

In at least one embodiment, the at least one sensor can further include a microphone to detect acoustic volume levels within the indoor space; and the at least one processor being operable to determine the occupancy or the airborne infection risk in the indoor space can be further based on the acoustic levels detected by the microphone.

In at least one embodiment, the at least one sensor can further include an ambient light sensor to detect indoor lighting in the indoor space; and the at least one processor being operable to determine the occupancy can be further based on the indoor lighting detected by the ambient light sensor.

In at least one embodiment, the at least one processor can be further operable to: compare the occupancy or the airborne infection risk to a respective pre-determined threshold value; and in response to determining that the occupancy or the airborne infection risk exceeds the respective pre-determined threshold value, generate a notification to be provided at a user interface.

In another broad aspect, a method for monitoring air in an indoor space is disclosed herein. The method can involve monitoring at least one of a carbon dioxide concentration or airborne particles in the indoor space for an extended time period to generate monitoring data. The extended time period can include at least one period when the indoor space is occupied and at least one period when the indoor space is unoccupied. The method can also involve operating at least one processor to receive the monitoring data; identify at least one region of interest in the monitoring data; and determine an air change rate for the indoor space based on the at least one region of interest.

In at least one embodiment, each region of interest identified in the monitoring data can include a portion of the monitoring data having a substantially constant rate of decay immediately followed by a portion of the monitoring data being substantially constant.

In at least one embodiment, for each region of interest, the monitoring data at the start of the region of interest can be greater than a pre-determined minimum value.

In at least one embodiment, for each region of interest, the monitoring data at the end of the region of interest can be greater than a pre-determined maximum value.

In at least one embodiment, the method can further involve detecting pressure changes within the indoor space; and operating the at least one processor to identify transitional ventilation events in the monitoring data based on the pressure changes detected; and identify the at least one region of interest in the monitoring data based on the transitional ventilation events.

In at least one embodiment, the method can further involve monitoring at least one of a carbon dioxide concentration or airborne particles in the indoor space in real-time to generate real-time monitoring data. The method can also involve operating the at least one processor to receive the real-time monitoring data; determine at least one of an occupancy in the indoor space based on the real-time monitoring data or an airborne infection risk in the indoor space based on the real-time monitoring data; and cause the occupancy or the airborne infection risk to be displayed at a user interface.

In at least one embodiment, the method can involve operating the at least one processor to determine a safe occupancy capacity based on the air change rate; and determine the airborne infection risk based on the safe occupancy capacity and the real-time monitoring data.

In at least one embodiment, the method can further involve detecting acoustic volume levels within the indoor space, wherein operating the at least one processor to determine the occupancy or the airborne infection risk in the indoor space can be further based on the acoustic levels detected.

In at least one embodiment, the method can further involve detecting indoor lighting in the indoor space, wherein operating the at least one processor to determine the occupancy can be further based on the indoor lighting detected.

In at least one embodiment, the method can further involve operating the at least one processor to compare the occupancy or the airborne infection risk to a pre-determined threshold value; and in response to determining that the occupancy or the airborne infection risk exceeds the pre-determined threshold value, generate a notification to be provided at a user interface.

### BRIEF DESCRIPTION OF THE DRAWINGS

Several embodiments will now be described in detail with reference to the drawings, in which:

FIG. 1 a block diagram of components interacting with an example monitoring device in accordance with an example embodiment;

FIG. 2 is a block diagram of components interacting with an example management system in accordance with an example embodiment;

FIG. 3A is an illustration of an example monitoring device, in accordance with an example embodiment;

FIG. 3B is an image of an example monitoring device, in accordance with another example embodiment;

FIG. 4 is an illustration of an example monitoring device within an indoor space, in accordance with an example embodiment;

FIG. 5 is a flowchart of an example method of monitoring indoor air, in accordance with an example embodiment;

FIG. 6A is an illustration of example monitoring data obtained over an extended period, in accordance with an example embodiment;

FIG. 6B is an illustration of example monitoring data obtained over an extended period, in accordance with another example embodiment;

FIG. 7 is an illustration of a plurality of example monitoring devices within an indoor space, in accordance with an example embodiment;

FIG. 8 is a flowchart of an example method of determining occupancy in an indoor space, in accordance with an example embodiment;

FIG. 9 is a flowchart of an example method of determining airborne infection risk in an indoor space, in accordance with an example embodiment;

FIG. 10 is an illustration of an example user interface for displaying monitoring data, in accordance with an example embodiment; and

FIG. 11 is an illustration of an example user interface for displaying airborne infection risk, in accordance with another example embodiment.

The drawings, described below, are provided for purposes of illustration, and not of limitation, of the aspects and features of various examples of embodiments described herein. For simplicity and clarity of illustration, elements shown in the drawings have not necessarily been drawn to scale. The dimensions of some of the elements may be exaggerated relative to other elements for clarity. It will be appreciated that for simplicity and clarity of illustration, where considered appropriate, reference numerals may be repeated among the drawings to indicate corresponding or analogous elements or steps.

### DESCRIPTION OF EXAMPLE EMBODIMENTS

The various embodiments described herein generally relate to methods (and associated systems configured to implement the methods) of monitoring indoor air. Indoor air can include pollutants, such as carbon dioxide or pathogens, exhaled by occupants in an indoor space. The systems, devices, and methods described herein can be used to determine occupancy in an indoor space. Furthermore, the systems, devices, and methods described herein can be used to determine airborne infection risk in an indoor space. The systems, devices, and methods described herein can also be used to characterize human activity in an indoor space.

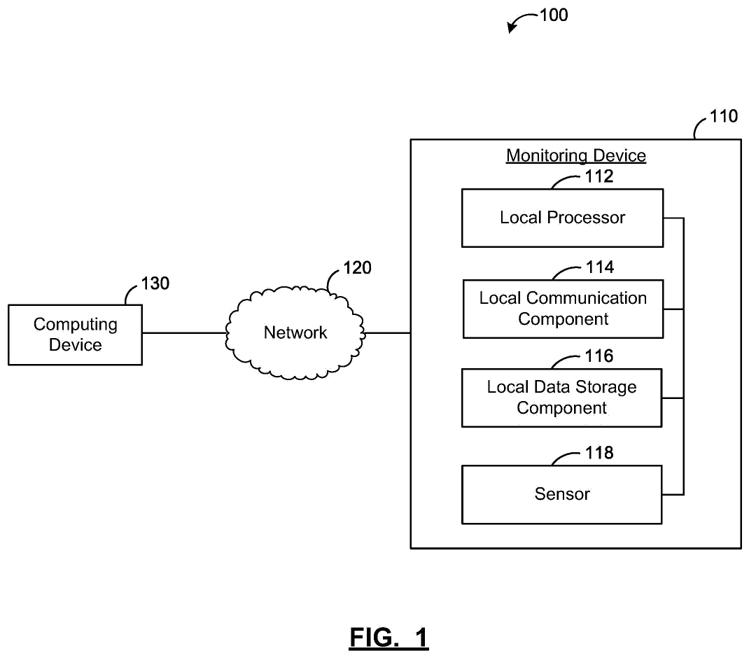

Reference is now made to FIG. 1, which illustrates a block diagram 100 of components interacting with an example monitoring device 110. As shown in FIG. 1, the monitoring device 110 can be in communication with a network 120.

The monitoring device 110 can be located within an indoor space for monitoring indoor air. The monitoring device 110 can include any networked device operable to connect to the network 120. A networked device is a device capable of communicating with other devices through a network such as the network 120. A networked device may couple to the network 120 through a wired or wireless connection. Although only one monitoring device 110 is shown in FIG. 1, it will be understood that more monitoring devices can connect to the network 120. Furthermore, although monitoring device 110 is shown in FIG. 1 as being in communication with network 120, in some embodiments, monitoring device 110 is not in communication with network 120.

The monitoring device 110 includes a local processor 112, a local communication component 114, a local data storage component 116, and a sensor 118. The local processor 112, the local communication component 114, and the local data storage component 116 can be combined into a fewer number of components or can be separated into further components. The local processor 112, the local communication component 114, and the local data storage component 116 may be implemented in software or hardware, or a combination of software and hardware.

The local processor 112 can operate to control the operation of the monitoring device 110. The local processor 112 can initiate and manage the operations of each of the other components within the monitoring device 110. The local processor 112 may be any suitable processors, controllers or digital signal processors that can provide sufficient processing power depending on the configuration, purposes and requirements of the monitoring device 110. In some embodiments, the local processor 112 can include more than one processor with each processor being configured to perform different dedicated tasks.

The local communication component 114 may include any interface that enables the monitoring device 110 to communicate with other devices and systems. In some embodiments, the local communication component 114 can include at least one of a serial port, a parallel port, a USB port, or a switch. Switches, including but not limited to dry contact relays, can be used to signal or activate other devices or systems. The local communication component 114 may also include at least one of an Internet, Local Area Network (LAN), Ethernet, Firewire, modem or digital subscriber line connection. Various combinations of these elements may be incorporated within the local communication component 114.

For example, the local communication component 114 may receive input from various input devices, such as a touch screen, a keypad, a thumbwheel, a track-ball, a card-reader, voice recognition software and the like depending on the requirements and implementation of the monitoring device 110.

The local data storage component 116 can include RAM, ROM, one or more hard drives, one or more flash drives or some other suitable data storage elements such as disk drives, etc. The local data storage component 116 can be removable. The local data storage component 116 can include one or more databases for storing information relating to the indoor space (e.g., geometry of indoor space including area and height, or volume of the indoor space), calibration data for sensor 118, and monitoring data generated by sensor 118.

The sensor 118 can be any device that detects its environment and generates corresponding data. For example, the sensor 118 can be a gas sensor, a particle sensor, a light sensor, a temperature sensor, a pressure sensor, a humidity sensor, an acoustic sensor, or any combination thereof. As a more specific example, the sensor 118 can be a carbon dioxide gas sensor to detect carbon dioxide concentration levels within the indoor space. As another example, the sensor 118 can include a particle sensor (e.g., PM2.5 sensor) to detect airborne particles within the indoor space. As a further example, the sensor 118 can be an ambient light sensor to detect indoor lighting being on or off. As another example, the sensor 118 can be a temperature sensor to detect the temperature within the indoor space. As a further example, the sensor 118 can be a barometric pressure sensor to detect pressure changes within the indoor space. As another example, the sensor 118 can be a microphone to detect acoustic volume levels within the indoor space. Although only one sensor 118 is shown in FIG. 1, it will be understood that more sensors can be included in the monitoring device 110.

The computing device 130 can include any networked device operable to connect to the network 120. Although only one computing device 130 is shown in FIG. 1, it will be understood that fewer or more computing devices 130 can connect to the network 120.

The computing device 130 may include at least a processor and memory, and may be a ventilation device, air filtration device, electronic tablet device, personal computer, workstation, server, portable computer, mobile device, personal digital assistant, laptop, smart phone, interactive television, video display terminals, gaming consoles, routers, smart speakers, and portable electronic devices or any combination of these.

The network 120 may be any network capable of carrying data, including the Internet, Ethernet, plain old telephone service (POTS) line, public switch telephone network (PSTN), integrated services digital network (ISDN), digital subscriber line (DSL), coaxial cable, fiber optics, satellite, mobile, wireless (e.g. Wi-Fi, WiMAX, Ultra-wideband, Bluetooth®), SS7 signaling network, fixed line, local area network, wide area network, and others, including any combination of these, capable of interfacing with, and enabling communication between the monitoring device 110, the computing device 130, and other networked devices.

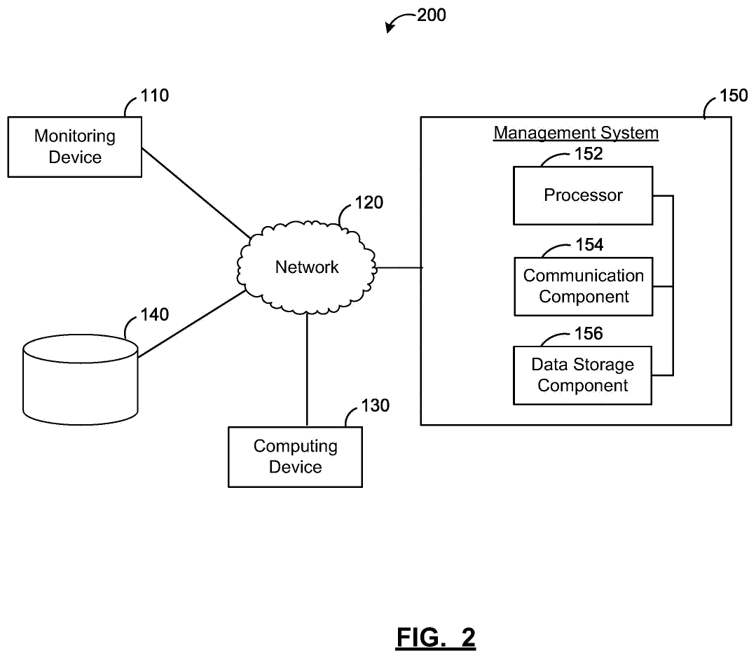

Reference is now made to FIG. 2, which illustrates a block diagram 200 of components interacting with an example management system 150. As shown in FIG. 1, the management system 150 is in communication with monitoring device 110, computing device 130, and an external data storage 140 via network 120. Although only one network 120 is shown in FIG. 2, it will be understood that more networks can be included. For example, the management system 150 can be in communication with the monitoring device 110 via a first network and the computing device 130 via a second network.

In at least one embodiment, the management system 150 is located in proximity to, or local to the indoor space. In at least one embodiment, the management system 150 is located remotely from the monitoring device 110.

In at least one embodiment, the management system 150 can be in communication with one or more monitoring devices 110 in an indoor space. In at least one embodiment, the management system 150 can be in communication with a plurality of monitoring devices 110 in a plurality of indoor spaces. In some embodiments, the plurality of indoor spaces can be located at a same site or venue (e.g., local to one another). In some embodiments, the plurality of indoor spaces can be located at different sites or venues (e.g., remote from one another).

The management system 150 includes a management processor 152, a management communication component 154, and a management data storage component 156. The management system 150 can be provided on one or more computer servers that may be distributed over a wide geographic area and connected via the network 120.

The management system 150 can perform various functions related to managing the monitoring device 110. The management system 150 can provide configuration data to the monitoring device 110, obtain monitoring data from the monitoring device 110, and/or process the monitoring data received from the monitoring device 110. Monitoring data can be automatically transmitted from the monitoring device 110 to the management system 150 via network 120. Monitoring data can also be manually transferred from the monitoring device 110 to the management system 150 via a removable local data storage component 116. Monitoring data can be transferred from the monitoring device 110 to the management system 150 via the computing device 130. For example, monitoring data can be transferred from the monitoring device 110 to the computing device 130 using a different network such as Bluetooth® and the computing device 130 can transfer the monitoring data to the management system 150 via network 120. In another example, the monitoring device 110 can display the monitoring data and the computing device 130 can scan the monitoring data transfer to the management system 150 via network 120.

The management processor 152, the management communication component 154, and the management data storage component 156 can be combined into a fewer number of components or can be separated into further components. The management processor 152, the management communication component 154, and the management data storage component 156 may be implemented in software or hardware, or a combination of software and hardware.

The management processor 152 can operate to control the operation of the management system 150. The management processor 152 can initiate and manage the operations of each of the other components within the management system 150. The management processor 152 may be any suitable processors, controllers or digital signal processors that can provide sufficient processing power depending on the configuration, purposes and requirements of the management system 150. In some embodiments, the management processor 152 can include more than one processor with each processor being configured to perform different dedicated tasks.

The management communication component 154 may include any interface that enables the management system 150 to communicate with other devices and systems. In some embodiments, the management communication component 154 can include at least one of a serial port, a parallel port, or a USB port. The management communication component 154 may also include at least one of an Internet, Local Area Network (LAN), Ethernet, Firewire, modem or digital subscriber line connection. Various combinations of these elements may be incorporated within the management communication component 154.

For example, the management communication component 154 may receive input from one or more input devices, such as a mouse, a keyboard, a touch screen, a button, a thumbwheel, a track-pad, a track-ball, a card-reader, voice recognition software and the like depending on the requirements and implementation of the management system 150.

Similar to the local data storage component 116, the management data storage component 156 can and the external data storage 140 can also include RAM, ROM, one or more hard drives, one or more flash drives or some other suitable data storage elements such as disk drives, etc.

Also similar to the local data storage component 116, the management data storage component 156 and the external data storage 140 can include one or more databases for storing information relating to the indoor air spaces (e.g., geometry of indoor space including area and height, or volume) and monitoring data of each of the indoor spaces (e.g., carbon dioxide concentration, audio volume, temperature, humidity, pressure, raw particle count, particle concentration, and lighting).

It will be understood that some components of FIG. 2, such as components of the management system 150 or the external data storage 140, can be implemented in a cloud computing environment.

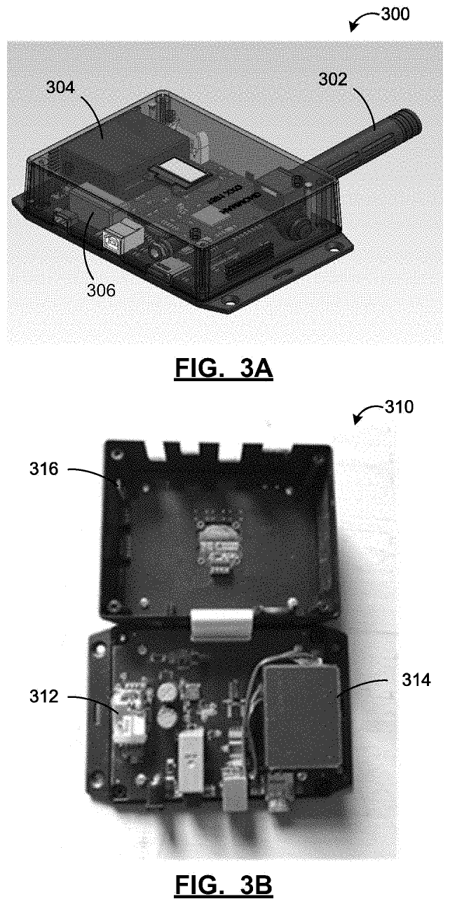

Reference is now made to FIG. 3A, which illustrates an example monitoring device 300, in accordance with at least one embodiment. As shown in FIG. 3A, monitoring device 300 can include a plurality of sensors such as a carbon dioxide gas sensor 302 and a particle sensor 304. Monitoring device 300 also includes a dry contact relay 306 for communication with other devices or systems.

Reference is now made to FIG. 3B, which shows another example monitoring device 310, in accordance with at least one embodiment. As shown in FIG. 3B, monitoring device 300 can also include a plurality of sensors such as a dual beam carbon dioxide gas sensor 312, a particle sensor 314, a temperature/humidity sensor 316, an acoustic sensor (not visible), and a light sensor (not visible). It will be understood FIGS. 3A and 3B are shown for illustrative purposes only and that fewer or more sensors can be included in example monitoring devices 300 and 310. For example, the temperature/humidity sensor 316 can include a barometer.

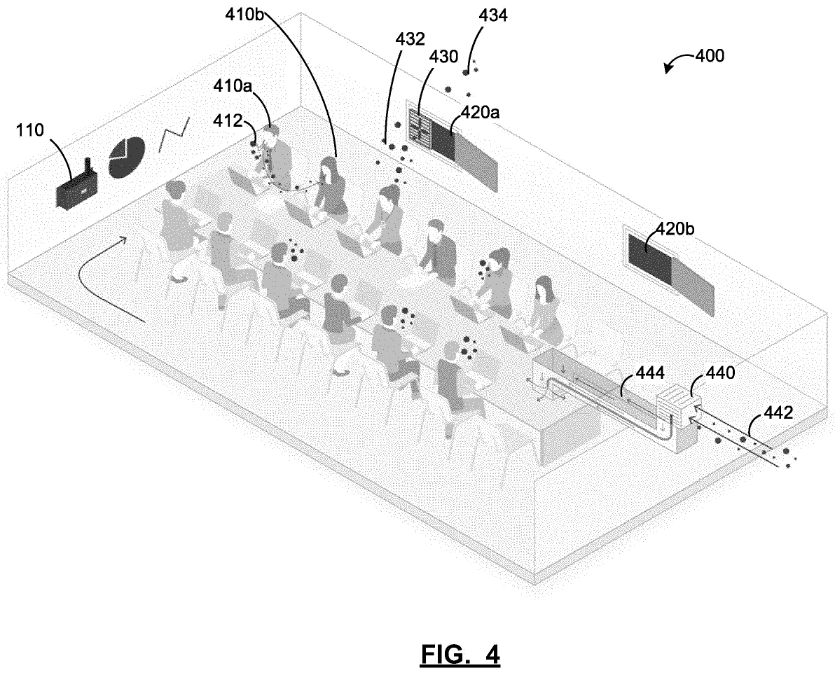

Reference is now made to FIG. 4, which illustrates monitoring device 110 within in an indoor space 400. People in the indoor space 400 may be infected with airborne diseases and shed pathogens (e.g., viral particles) that contaminate the indoor air. As illustrated in FIG. 4, someone 410a with an infection may exhale infected particles 412 (e.g., pathogens) and another person 410b can breathe in the infected particles 412. In this manner, airborne infection can be transmitted between occupants.

To reduce the risk that someone inhales infected particles, and thereby reduce the airborne infection risk, ventilation and/or filtration can be provided to the indoor space 400. Ventilation can dissipate the concentration of air contaminants by removing indoor air and adding fresh air. Filtration can reduce the concentration of air contaminants by removing air contaminants from indoor air before it is recirculated and/or fresh air before it is added. Ventilation devices can include natural air ventilation devices as well as forced air circulation devices. For example, windows 420a, 420b and doors (not shown in FIG. 4) provide natural air ventilation by allowing for the exchange of indoor air and fresh, outdoor air. Forced air circulation devices may or may not include filtration components.

Forced air circulation devices can include a fan, such as example fan 430, and/or a heating, ventilation, and air conditioning system (HVAC), such as example HVAC 440. Although fan 430 is shown as being a window-mounted fan, it will be understood that fan 430 may not be window-mounted. Fan 430 can operate in an exhaust mode to draw air 432 from the indoor space 400 and push air 434 to the outdoors. In contrast, HVAC 440 can draw fresh air 442 from outside, filter the fresh air, and push the clean, fresh air 444 to the indoor space 400. It should be noted that fan 430 can also operate in an intake mode to draw air from outside and into the indoor space 400. As well, HVAC 440 can also draw air 432 from the indoor space 400, filter the air, and return the air to the indoor space 400.

Monitoring device 110 can be provided in indoor space 400 to monitor air in the indoor space 400. More specifically, monitoring device 110 can be used to monitor carbon dioxide concentration in the indoor space 400. Since people 410a, 410b expel carbon dioxide as they breathe, speak, cough, or sneeze, carbon dioxide concentration can be used as a proxy for the concentration of exhaled air of occupants. Accordingly, monitoring carbon dioxide concentration levels can be indicative of the number of occupants in the indoor space 400, the ventilation in the indoor space 400, and further, whether the ventilation is sufficient for the number of occupants.

In some cases, the ventilation may be low, that is the amount of fresh air 442 brought into the indoor space 400 may be low, but the ventilation system may be continuously filtering airborne particles (aerosols) from the indoor air 400. Monitoring device 110 can be used to monitor particle concentration in the indoor space 400. Decreases in particle concentration levels can be indicative of the air filtration in the indoor space 400, and further whether the filtration is sufficient for the number of occupants.

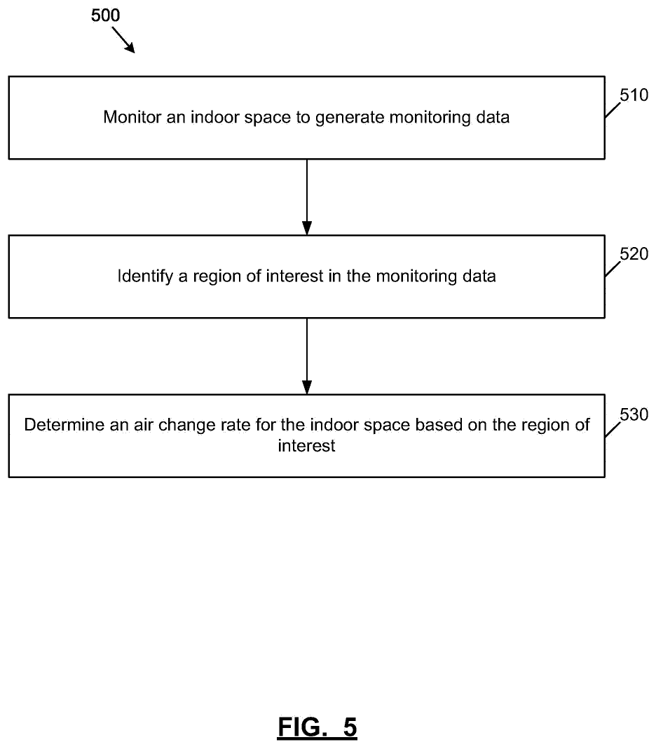

Reference is now made to FIG. 5, which shows an example method 500 of monitoring indoor air in a flowchart diagram. To assist with the description of the method 500, reference will be made simultaneously to FIG. 1 and FIG. 4. A device, such as monitoring device 110 in block diagram 100, can be configured to implement method 500. The monitoring device 110 is located in an indoor space, such as indoor space 400.

Method 500 can begin at 510. The monitoring device 110 can monitor the indoor air within the indoor space 400 and generate monitoring data. The monitoring data can be stored in local data storage component 116.

In at least one embodiment, monitoring the indoor air at 510 can involve monitoring carbon dioxide concentration levels and the monitoring data can include carbon dioxide concentration data. Furthermore, monitoring the indoor air at 510 can involve injection of a tracer gas to the indoor space to artificially elevate carbon dioxide concentration in the indoor space. The monitoring device 110 can measure the carbon dioxide concentration in the indoor air as it is ventilated and the carbon dioxide concentration decays to a steady state, or ambient carbon dioxide concentration levels.

In at least one embodiment, monitoring the indoor air at 510 may not involve injection of a tracer gas. Instead, the indoor air can be monitored at 510 for an extended period. The extended period includes periods when the space is occupied and periods when the space is unoccupied. For example, the extended period can be about 5 days to 10 days. As a more specific example, the indoor air can be monitored for about 7 days. As a result, the monitoring data can include periods of elevated carbon dioxide concentration levels (i.e., occupied) and periods of ambient carbon dioxide concentration levels (i.e., unoccupied).

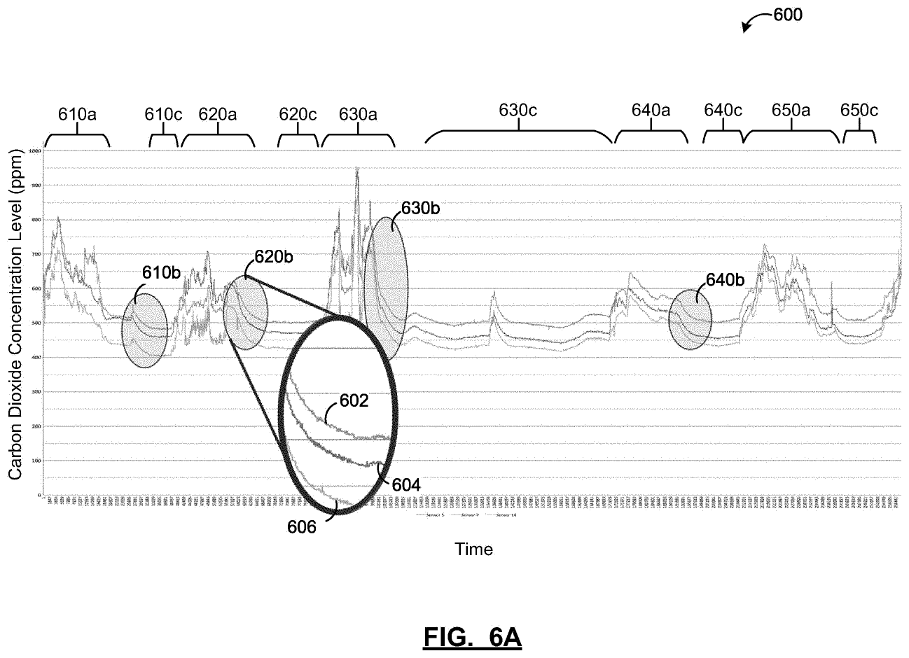

FIG. 6A shows an example chart 600 of example monitoring data obtained over an extended period in 510. In this example, three monitoring devices 110 are used to monitor indoor air for carbon dioxide concentration levels within an indoor space, shown by monitoring data 602, 604, and 606. As can be seen in chart 600, the indoor space 400 is monitored over periods of elevated carbon dioxide concentration levels 610a, 620a, 630a, 640a, and 650a as well as periods of ambient carbon dioxide concentration levels 610c, 620c, 630c, 640c, and 650c.

At 520, the monitoring device 110 can identify a region of interest in the monitoring data obtained at 510. The region of interest is a subset of the monitoring data and relates to a transition from the elevated levels to a steady state, or ambient level. The region of interest can include a start time (tₛₜₐᵣₜ), an end time (t_{end}), a starting concentration level (Cₛₜₐᵣₜ), and an ending concentration level (C_{end}).

When a tracer gas is used at 510, the region of interest can be more readily identified as the monitoring data contains only one transition from the artificially elevated carbon dioxide concentration level to a steady state, or an ambient carbon dioxide concentration level.

When a tracer gas is not used (i.e., monitoring at 510 over an extended period), the monitoring data contains several transitions 610b, 620b, 630b, and 640b from elevated carbon dioxide concentration levels 610a, 620a, 630a, 640a, and 650a to ambient carbon dioxide concentration levels 610c, 620c, 630c, 640c, and 650c. The transition, that is, the decay of carbon dioxide concentration levels occurs as the HVAC 440 pulls out old air and inserts fresh air to the indoor space 400.

Various algorithms can be used to identify the regions of interest 610b, 620b, 630b, and 640b. In at least one embodiment, a neural network can be trained to identify the regions of interest 610b, 620b, 630b, and 640b. In at least one embodiment, regions of interest 610b, 620b, 630b, and 640b can identified based on a portion of the monitoring data having a substantially constant rate of decay (e.g., negative slope) followed by a portion of the monitoring data being substantially constant (e.g., zero slope). The portions of the monitoring data being substantially constant relates to a steady state in the concentration level.

In at least one embodiment, identification of the regions of interest 610b, 620b, 630b, and 640b can require that the starting carbon dioxide concentration levels 610a, 620a, 630a, 640a, and 650a and the ending carbon dioxide concentration levels 610c, 620c, 630c, 640c, and 650c satisfy pre-determined minimum and maximum values, respectively. For example, the starting carbon dioxide concentration levels 610a, 620a, 630a, 640a, and 650a may need to be at least 2000 ppm. As another example, the ending carbon dioxide concentration levels 610c, 620c, 630c, 640c, and 650c may need to decay at least to 37% of the corresponding starting carbon dioxide concentration levels 610a, 620a, 630a, 640a, and 650a.

At 530, the monitoring device 110 can determine an air change rate of the indoor space 400 based on a region of interest identified at 520.

In some embodiments, the air change rate can be determined based on equation (1) as follows:

$$ACH = \frac{- 1 \times ln ( \frac{{c}_{end} - {c}_{ambient}}{{c}_{start} - {c}_{ambient}} )}{{t}_{end} - {t}_{ambient}} \qquad Eq . ( 1 )$$

where:
   tₛₜₐᵣₜ is the start time of the region of interest;
   t_{end} is the end time of the region of interest;
   Cₛₜₐᵣₜ is the starting concentration level of the region of interest;
   C_{end} is the ending concentration level of the region of interest; and
   C_{ambient} is an ambient concentration level.

In at least one embodiment, the ambient concentration level (C_{ambient}) can be an ambient concentration level measured outdoors. In other embodiments, ambient concentration level (C_{ambient}) can be measured in the indoor space. Ambient indoor concentration levels can differ from ambient outdoor concentration levels. That is, ambient indoor concentration levels may not equalize with ambient outdoor concentration levels. As such, ambient indoor concentration levels can be higher than ambient outdoor concentration levels even after long periods of time. Use of an ambient indoor concentration level as the ambient concentration level (C_{ambient}) in Eq. (1) can provide a more accurate estimation of the air change rate.

When a tracer gas is not used (i.e., monitoring at 510 over an extended period), an air change rate can be determined for each region of interest 610b, 620b, 630b, and 640b and an average of the air change rates over the extended period can be determined to obtain an average air change rate for the monitoring device 110.

In at least one embodiment, monitoring at 510 can involve monitoring particle concentration levels over an extended period and the real-time monitoring data can include particle concentration data. As a result, the monitoring data can include periods of elevated particle concentration levels and periods of ambient particle concentration levels (i.e., particles settled or filtered by HVAC 440 during vacancy).

During ambient periods, when few particles are being disturbed and few new particles are being generated, the expected rate of decrease in the particle concentration level due to natural particle removal rates for different particle sizes at different rates can be modeled.

Elevated particle concentration levels can result from particles being agitated and/or generated during occupancy. In particular, large spikes in particle concentration levels can result from certain activities such as cooking or frying. The decay of particle concentration levels following such large spikes can used to estimate the air filtration provided to the indoor space. Particle removal rates greater than the natural removal rate (determined from ambient periods) can be attributed to filtration.

Particles of different sizes can be removed at different levels of filtration, such as settling, diffusion, impaction and electrostatic deposition. However, small particles can remain suspended in the air for extended periods, depending on the humidity, air turbulence, and various other factors. In some cases, particles smaller than 0.5 micrometers (μm) can remain suspended in the air for more than 24 hours. Furthermore, once small particles adhere to surfaces, they can be the most challenging to dislodge.

FIG. 6B shows an example chart 660 of example monitoring data obtained over an extended period in 510 in another indoor space. In this example, a monitoring device 110 is used to monitor indoor air for particle concentration levels within a unit of a residential apartment building. In this example, the monitoring device 110 can detect particles greater than 0.3 micrometers (μm). Detection of this size is useful because such particles remain suspended the longest.

As shown in chart 660, large spikes in particle concentration levels 662a, 662b, and 662c can occur during the monitoring period. Two of the three spikes were due to cooking in the monitored unit; while a third spike was due to cooking in another unit that shares the same ventilation system. As can be seen, small particles can spread widely.

In at least one embodiment, the determination of the air change rate can be based on both carbon dioxide concentration levels and particle concentration levels.

In at least one embodiment, the monitoring device 110 can also monitor the barometric pressure within the indoor space 400 over the extended period. That is, the monitoring data can include pressure data. Pressure data can be correlated to transitional ventilation events observed in the carbon dioxide concentration data.

Transitional ventilation events can result in abrupt changes in the carbon dioxide concentration levels and pressure in the indoor space 400. Transitional ventilation events include, for example, windows or doors opening or closing, or the HVAC 440 changing speed, including starting up or shutting down. Such events can result in a sudden change in carbon dioxide concentration levels or the rate of change in carbon dioxide concentration levels, as well as a sudden change in pressure. In some cases, sudden changes in pressure may not be observed with transitional ventilation events if the indoor space 400 is not sealed.

The regions of interest 610b, 620b, 630b, and 640b can be determined based in part on the pressure data. For example, if the HVAC 440 is expected to shutdown, the shutdown event can be identified by a corresponding change in pressure. In turn, the region of interests 610b, 620b, 630b, and 640b can be identified as being prior to the time at which a change in pressure is observed. Determining the regions of interest 610b, 620b, 630b, and 640b in part on the pressure data can improve the accuracy of the resultant air change rate.

Method 500 allows for the air change rate of the indoor space to be determined remotely. That is, by monitoring the indoor air over an extended period without the use of a tracer gas at 510, method 500 allows for the air change rate to be determined without visiting the space.

Method 500 can be implemented by monitoring device 110. That is, the local processor 112 can be configured to store the monitoring data of 510 in the local data storage component 116, identify the regions of interest at 520, and determine the air change rate at 530.

In some embodiments, method 500 can be implemented by monitoring device 110 and another networked device, such as computing device 130 or management system 150. For example, computing device 130 and/or management system 150 can receive the regions of interest at 520 and determine the air change rate at 530. In another example, computing device 130 and/or management system 150 can receive the monitoring data of 510 and identify the regions of interest at 520 and the air change rate at 530. The monitoring data can be stored in the management data storage component 156 and/or the external data storage 140. Other embodiments are possible.

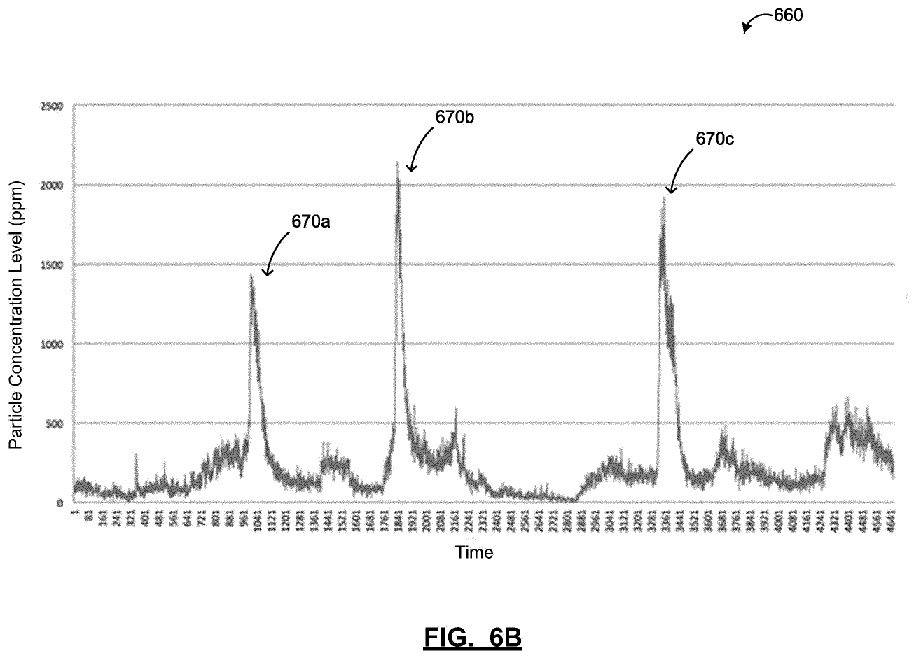

Implementation of method 500 involving computing device 130 and/or management system 150 can be appropriate particularly when a plurality of monitoring devices is used within an indoor space. FIG. 7 shows an example illustration 700 of a plurality of monitoring devices 110a, 110b, 110c, 110d, 110e, 110f, and 110g within an indoor space. The indoor space includes a plurality of areas 702, 704, and 706. Each area is monitored by one or more monitoring devices located therein. For example, monitoring devices 110a, 110b are located within area 702; monitoring devices 110c, 110d, 110e, and 110f are located within area 704; and monitoring devices 110g is located within area 706.

In at least one embodiment, an air change rate can be determined for each monitoring device 110a, 110b, 110c, 110d, 110e, 110f, and 110g. An average of the air change rates for the one or more monitoring devices in an area can be determined to obtain an air change rate for each area 702, 704, and 706. For example, the air change rate of area 702 can be determined from the air change rate of monitoring devices 110a and 110b; the air change rate of area 704 can be determined from the air change rate of monitoring devices 110c, 110d, 110e, and 110f; and the air change rate of area 706 can be determined from the air change rate of monitoring device 110g. Furthermore, an air change rate for the indoor space can be determined from the air change rate of each area.

In at least one embodiment, the air change rate of each area can be converted to a rate of ventilation for that area. The rate of ventilation for an area can be determined based on the air change rate and the volume of the area. The volume of each area can be determined by measuring the dimensions, that is the floor area and ceiling height. In some cases, a rate of ventilation can be a better indicator than air change rate for whether ventilation is sufficient. For example, an area may have low air change rate due to a tall ceiling, and therefore a larger volume. However, the area may still have a sufficiently large quantity of air flow (i.e., rate of ventilation).

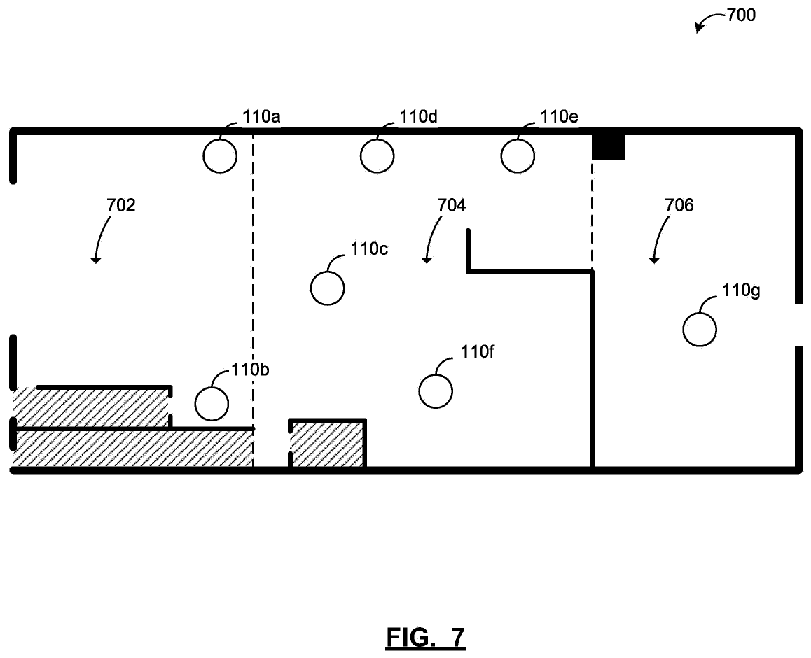

Reference is now made to FIG. 8, which shows an example method 800 of determining occupancy in an indoor space in a flowchart diagram. To assist with the description of the method 800, reference will be made simultaneously to FIG. 2. A system, such as the system in block diagram 200 having a monitoring device 110 and a management system 150 can be configured to implement the method 800. The monitoring device 110 is located in an indoor space, such as indoor space 400. In at least one embodiment, the management system 150 can be located remotely from the monitoring device 110.

Method 800 can begin at 810. At 810, an air change rate for the indoor space 400 can be obtained. Obtaining the air change rate for the indoor space can involve retrieving the air change rate from local data storage component 116, management data storage component 156, or external data storage 140. In at least one embodiment, the air change rate for the indoor space 400 can be obtained using method 500.

At 820, the monitoring device 110 can monitor the indoor space in real-time and generate real-time monitoring data. In at least one embodiment, monitoring at 820 can involve monitoring carbon dioxide concentration levels and the real-time monitoring data can include carbon dioxide concentration data. In at least one embodiment, the monitoring device 110 can monitor particle concentration levels and generate monitoring data including particle concentration data. In at least one embodiment, the monitoring device 110 can monitor additional properties of the indoor space and generate corresponding real-time monitoring data, including but not limited to raw particle count data, acoustic volume data, lighting data, pressure data, humidity data, and temperature data.

In at least one embodiment, the real-time monitoring data can be displayed on a user interface. In at least one embodiment, the user interface can be provided on the monitoring device 110. In other embodiments, the user interface can be provided on the computing device 130. The computing device 130 can be local, or remote from the indoor space 400. In at least one embodiment, the real-time monitoring data can be encoded and encoded data, such as a QR code, can be displayed. The computing device 130 can be used to scan the encoded data displayed on the monitoring device 110 and display the real-time monitoring data on the computing device 130. The computing device 130 can further transmit the real-time monitoring data to the management system 150.

At 830, the monitoring device 110 can determine an occupancy in the indoor space can based on the real-time monitoring data generated at 820 and the air change rate obtained for the indoor space at 810. The occupancy determined at 830 can also be displayed on a user interface.

The determination of occupancy in the indoor space based on the air change rate for the indoor space determined at 810 allows for the ventilation system to be accounted for. Various methods can be used for determining occupancy based on carbon dioxide concentration levels. Some methods can be simplified by assuming a well-mixed room in a stable state.

For example, a percentage of the real-time monitoring data, that is, a percentage of the measured carbon dioxide concentration level can be attributed to occupants. The presence of exhaled air can be determined based on equation (2) as follows:

$$Percentage of Exhaled Air Present = \frac{{c}_{indoor} - {c}_{outdoor}}{{c}_{person} - {c}_{outdoor}} \qquad Eq . ( 2 )$$

where:
   C_{indoor} is the indoor carbon dioxide concentration level;
   Cₚₑᵣₛₒₙ is a person's average exhaled air carbon dioxide concentration; and
   C_{outdoor} is the ambient outdoor carbon dioxide concentration level.

A person's average carbon dioxide concentration in exhaled air at rest (Cₚₑᵣₛₒₙ) is generally consistent and can be approximately 38,500 ppm of carbon dioxide. As well, an ambient outdoor carbon dioxide concentration level (C_{outdoor}) is approximately 450 ppm. If the measured carbon dioxide level for the indoor space (C_{indoor}) is 1500 ppm, then approximately 2.76% of the carbon dioxide concentration level can be attributed to occupants.

If the indoor space has a volume of 100 cubic meters and an air change rate of 5, the rate of fresh air is approximately 500 cubic meters per hour (m³/h), or 8333 liters per minute (L/m) or 139 liters per second (L/s). Assuming that a steady state of carbon dioxide concentration level is achieved, exhaled air is generated by occupants at a rate of approximately 3.84 L/s (e.g., 139 L/s x 2.76%). If the average person breathes 6 L/m (0.1 L/s), then there can be approximately 38 people present (e.g., 3.84 L/s÷0.1 L/s). This determination of occupancy is provided for illustrative purposes only and it will be understood that other calculation methods are possible.

In at least one embodiment, an initial estimate of a number of occupants can be determined based on carbon dioxide concentration data and/or the particle concentration data. The initial estimate can be refined based on additional data. Additional data can include, but is not limited to, acoustic volume data and lighting data.

For example, higher acoustic volumes are typically associated with greater occupancy levels. A pre-determined acoustic volume range can be determined based the initial estimate of the number of occupants. The real-time acoustic volume data can be compared with the pre-determined acoustic volume range. If the real-time acoustic volume data falls outside of the pre-determined acoustic volume range, the initial estimate of the number of occupants can be modified accordingly.

As another example, indoor lighting being on is typically indicative of the presence of occupants and indoor lighting being off is typically indicative of the absence of occupants. The initial estimate of the number of occupants can be modified if the real-time indoor lighting data indicates the presence or absence of occupants.

In at least one embodiment, the occupancy in the indoor space can be compared with a pre-determined occupancy threshold. A notification can be generated when the number of occupants within the indoor space is greater than the pre-determined threshold. For example, the pre-determined occupancy threshold can relate to an occupancy limit due public health, fire safety, security, or any other measures. As a more specific example, an indoor space may be expected to be vacant overnight. If the indoor space is occupied (i.e., number of occupants greater than zero), the monitoring device 110 can generate a notification. The notification can be displayed on a user interface or transmitted to computing device 130 or management system 150.

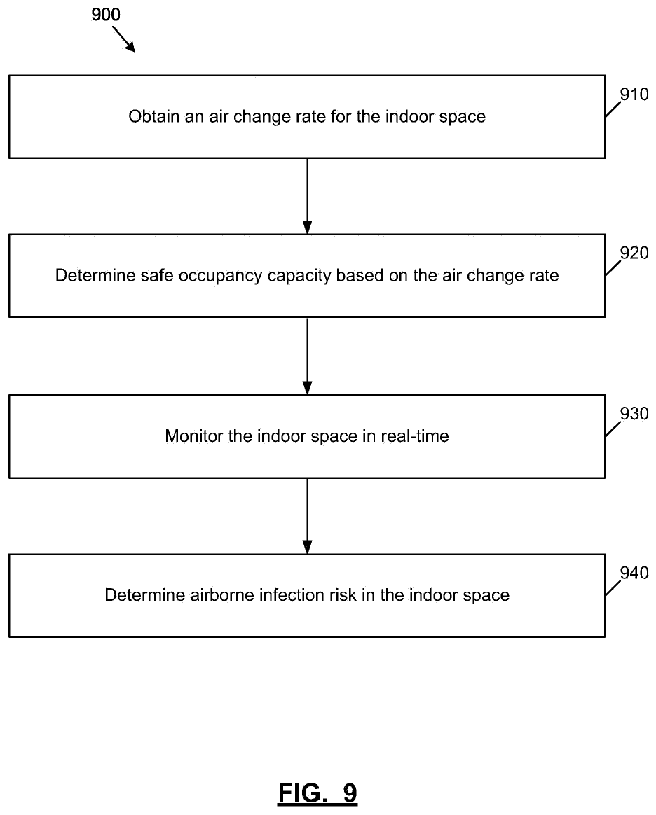

FIG. 10 illustrates an example user interface 1000. As shown in FIG. 10, the user interface 1000 can display current real-time monitoring data 1002, configuration settings, such as a setpoint 1004, and notifications 1006. Notifications can be generated based on the current real-time monitoring data 1002 and the configuration settings. As shown in FIG. 10, a notification 1006 can be displayed when the current real-time monitoring data 1002 exceeds the setpoint 1004. The notification 1006 can be configured to include an audio alert or annunciation, as indicated by alarm setting 1008. While the current real-time monitoring data 1002 and corresponding setpoint 1004 shown in example user interface 1000 relates to carbon dioxide concentration data, it will be understood that the current real-time monitoring data 1002 and corresponding setpoint 1004 can relate to other monitoring data. For example, the current real-time monitoring data 1002 can relate to the occupancy in the indoor space determined at 830 and the setpoint 1004 can relate to the pre-determined occupancy threshold.

In at least one embodiment, the computing device 130 and/or management system 150 can be configured to automatically undertake various operations in response to the notification. The computing device 130 can be a ventilation device that is automatically controlled when the occupancy reaches pre-determined occupancy thresholds. For example, the ventilation device can be but is not limited to fan 430 or HVAC 440. Automatic control can relate to starting, stopping, changing speeds, and/or changing modes of operation.

Method 800 can be implemented by monitoring device 110. That is, the local processor 112 can be configured to obtain the air change rate at 810, monitor the indoor space in real-time at 820, and determine the occupancy at 830.

In some embodiments, method 800 can be implemented by monitoring device 110 and another networked device, such as computing device 130 or management system 150. For example, computing device 130 and/or management system 150 can obtain the air change rate at 810, receive the monitoring data from the monitoring device 110 at 820, and determine the occupancy at 830. The air change rate at 810 can be stored in the management data storage component 156 and/or the external data storage 140. Other embodiments are possible.

Implementation of method 800 involving computing device 130 and/or management system 150 can be appropriate particularly when a notification is provided to computing device 130 or management system 150 and/or various operations are automatically undertaking in response to the notification.

In at least one embodiment, human activity in the indoor space can be characterized based on the carbon dioxide concentration levels given a fixed occupancy. People exhale higher concentrations of carbon dioxide with various types and levels of activities, such as sleeping or awake, at rest or aerobic activity, or talking or singing. Changes in carbon dioxide can also indicate a door or window opening.

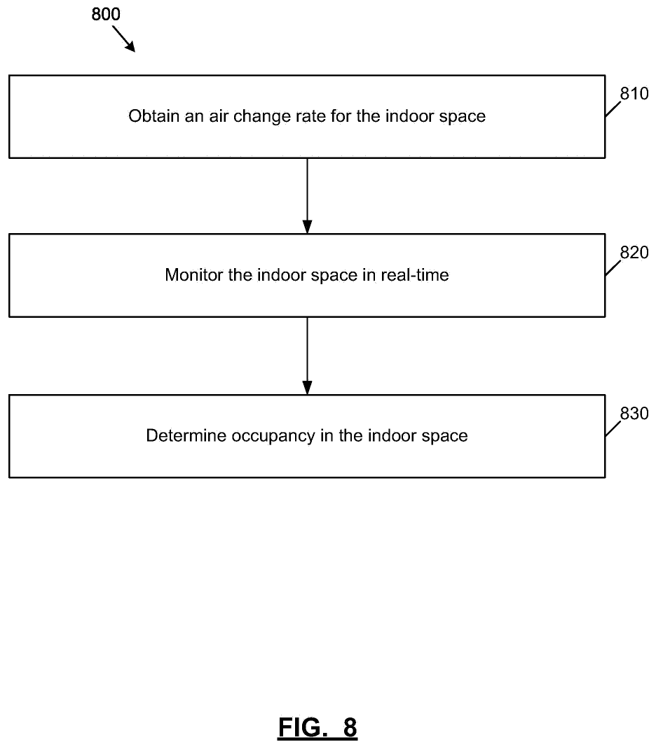

Reference is now made to FIG. 9, which shows an example method 900 of determining airborne infection risk in an indoor space in a flowchart diagram. To assist with the description of the method 900, reference will be made simultaneously to FIG. 2. A system, such as the system in block diagram 200 having a monitoring device 110 and a management system 150 can be configured to implement the method 900. The monitoring device 110 is located in the indoor space, such as indoor space 400. In at least one embodiment, the management system 150 can be located remotely from the monitoring device 110.

Method 900 can begin at 910. At 910, an air change rate for the indoor space can be obtained. Obtaining the air change rate for the indoor space can involve retrieving the air change rate from local data storage component 116, management data storage component 156, or external data storage 140. In at least one embodiment, the air change rate for the indoor space 400 can be obtained using method 500.

At 920, the monitoring device 110 can determine a safe occupancy capacity for the indoor space based on the air change rate obtained at 910. The safe occupancy capacity is aims to keep exhaled air concentration below certain levels. The safe occupancy capacity can be generated from on a pre-determined model or determined from look-up table.

At 930, the monitoring device 110 can monitor the indoor space in real-time and generate real-time monitoring data. In at least one embodiment, monitoring at 930 can involve monitoring carbon dioxide concentration levels and the real-time monitoring data can include carbon dioxide concentration data. In at least one embodiment, the monitoring device 110 can monitor particle concentration levels and generate monitoring data including particle concentration data. In at least one embodiment, the monitoring device 110 can monitor additional properties of the indoor space and generate corresponding real-time monitoring data, including but not limited to raw particle count data, acoustic volume data, lighting data, pressure data, humidity data, and temperature data.

At 940, the monitoring device 110 can determine airborne infection risk in the indoor space based on the real-time monitoring data generated at 930 and the safe occupancy capacity obtained for the indoor space at 920. The determination of airborne infection risk within the indoor space based on the air change rate for the indoor space determined at 910, via the safe occupancy capacity obtained at 920, allows for the ventilation system to be accounted for.

Various methods can be used for determining airborne infection risk. In at least one embodiment, the determination of the airborne infection risk can be determined based on models for aerosol disease transmission, such as the Wells-Riley model. It will be understood that other calculation methods are possible. For example, the Wells-Riley model for aerosol disease transmission include assumptions about air change rate and mixing. Instead, actual simulation of air flows for given floorplans and interiors can be used. As well, additional factors can be included such as outdoor temperatures for stack effect, doors, and/or windows.

It should be noted that the airborne infection risk is based on configuration settings, such as an infectiousness of the pathogen, a number of infected occupants, an activity type and level of infected occupants, and an average exposure time. The infectiousness of the pathogen can also depend on factors such as the temperature and humidity. Such configuration settings can be user-selectable and stored on in the local data storage component 116, management data storage component 156, and/or external data storage 140.

In at least one embodiment, an initial estimate of the airborne infection risk can be determined based on carbon dioxide concentration data and/or the particle concentration data. The initial estimate can be refined based on additional data. Additional data can include, but is not limited to, raw particle count data and acoustic volume data.

For example, raw particle count data, in contrast to particle concentration data, can provide insight on the filtering performance of the HVAC 440. The real-time raw particle count data measured by the monitoring device 110 relates to an indoor particle count and can be compared with an outdoor particle count. In at least one embodiment, the outdoor particle count is a pre-determined value. In some embodiments, the outdoor particle count is measured in real-time as well. The difference between the indoor particle count and the outdoor particle count reflects the filtering performance of the HVAC 440 and can be compared with a pre-determined threshold. If the difference between the between the indoor and outdoor particle counts is greater than the pre-determined threshold, the initial estimate of the airborne infection risk can be reduced accordingly.

As another example, acoustic volume data can be correlated to particular human activities that are associated with higher risks of airborne infection. People exhale higher concentrations of potentially infectious particles when they sing, yell, or shout compared to not speaking. Pre-determined acoustic volume ranges can be associated with such higher risk activities. The real-time acoustic volume data can be compared with the pre-determined acoustic volume ranges. If the real-time acoustic volume data falls within the pre-determined acoustic volume ranges, the initial estimate of the airborne infection risk can be increased accordingly.

The safe occupancy capacity of 920, the real-time monitoring data of 930, and/or the airborne infection risk of 940 can be displayed on a user interface. In at least one embodiment, the user interface can be provided on the monitoring device 110. In other embodiments, the user interface can be provided on computing device 130. The computing device 130 can be local, or remote from the indoor space 400.

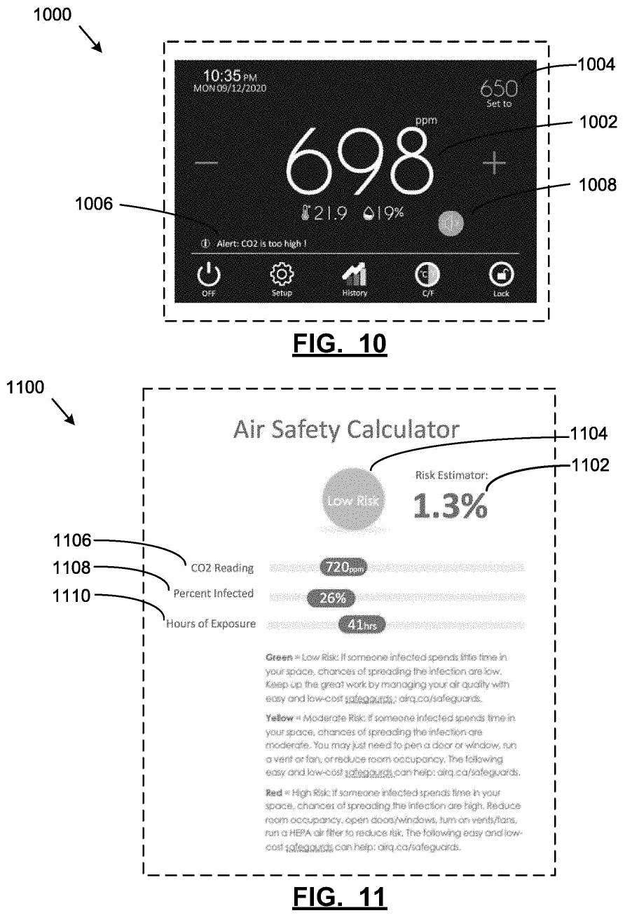

Reference is now made to FIG. 11, which illustrates another example user interface 1100. As shown in FIG. 11, the user interface 1100 displays the airborne infection risk, which can be indicated by a percentage 1102 or as a color-coded risk level 1104. The user interface 1100 also displays current real-time monitoring data, such as carbon dioxide concentration data 1106, and configuration settings, such as a number of infected occupants 1108 and an average exposure time 1110.

In at least one embodiment, a computing device 130 can access information about the airborne infection risk of an indoor space prior to entering the indoor space. Each indoor space can be associated with one or more indoor space identifiers that are unique to the indoor space, such as a QR code, barcode, or other identification code. The indoor space identifier can be provided outside of the indoor space, either virtually (e.g., online) or on physical signage. The computing device 130 can be configured to automatically request and receive information via a web-based graphical user interface about the airborne infection risk of the indoor space by scanning the QR code for the indoor space. The information about the airborne infection risk of an indoor space can include real-time monitoring data, as well as historical monitoring data.

Permission settings can be used to control the information accessible to computing device 130. That is different information can be made available to a computing device 130, depending on the type of access associated with the computing device 130. For example, information available to a computing device 130 having public access may be more limited than information available to a computing device 130 having private (e.g., management or employee) access.

In at least one embodiment, different indoor space identifiers can be provided for different permission settings. For example, a first QR code can be provided for public access, such as outside of the indoor space, and a second QR code that is separate and distinct from the first QR code can be provided for private access, such as on the monitoring device 110 itself.

In at least one embodiment, when the management system 150 is in communication with a plurality of monitoring devices 110, the airborne infection risk of the different monitoring devices can be displayed on a graphical map. For example, when the monitoring devices 110 are located at different sites or venues, such a graphical map can allow public users to plan a trip route and destinations. As another example, when the monitoring devices 110 are located within the same site or venue, such a restaurant, library, or gym, such a graphical map can allow users to plan table or space allocation.

In at least one embodiment, a number of occupants can be another configuration setting. The monitoring device 110 can estimate the carbon dioxide concentration level for the given number of occupants and use the estimated carbon dioxide concentration level for determining the airborne infection risk at 940 instead of real-time carbon dioxide concentration data.

It will be appreciated that numerous specific details are set forth in order to provide a thorough understanding of the example embodiments described herein. However, it will be understood by those of ordinary skill in the art that the embodiments described herein may be practiced without these specific details. In other instances, well-known methods, procedures and components have not been described in detail so as not to obscure the embodiments described herein. Furthermore, this description and the drawings are not to be considered as limiting the scope of the embodiments described herein in any way, but rather as merely describing the implementation of the various embodiments described herein.

It should be noted that terms of degree such as “substantially”, “about” and “approximately” when used herein mean a reasonable amount of deviation of the modified term such that the end result is not significantly changed. These terms of degree should be construed as including a deviation of the modified term if this deviation would not negate the meaning of the term it modifies.

In addition, as used herein, the wording “and/or” is intended to represent an inclusive-or. That is, “X and/or Y” is intended to mean X or Y or both, for example. As a further example, “X, Y, and/or Z” is intended to mean X or Y or Z or any combination thereof.

It should be noted that the term “coupled” used herein indicates that two elements can be directly coupled to one another or coupled to one another through one or more intermediate elements.

The embodiments of the systems and methods described herein may be implemented in hardware or software, or a combination of both. These embodiments may be implemented in computer programs executing on programmable computers, each computer including at least one processor, a data storage system (including volatile memory or non-volatile memory or other data storage elements or a combination thereof), and at least one communication interface. For example and without limitation, the programmable computers (referred to below as computing devices) may be a server, network appliance, embedded device, computer expansion module, personal computer, laptop, personal data assistant, cellular telephone, smart-phone device, tablet computer, router, smart speaker, ventilation device, air filtration device, wireless device or any other computing device capable of being configured to carry out the methods described herein.

In some embodiments, the communication interface may be a network communication interface. In embodiments in which elements are combined, the communication interface may be a software communication interface, such as those for inter-process communication (IPC). In still other embodiments, there may be a combination of communication interfaces implemented as hardware, software, and combination thereof.

Program code may be applied to input data to perform the functions described herein and to generate output information. The output information is applied to one or more output devices, in known fashion.

Each program may be implemented in a high level procedural or object oriented programming and/or scripting language, or both, to communicate with a computer system. However, the programs may be implemented in assembly or machine language, if desired. In any case, the language may be a compiled or interpreted language. Each such computer program may be stored on a storage media or a device (e.g., ROM, magnetic disk, optical disc) readable by a general or special purpose programmable computer, for configuring and operating the computer when the storage media or device is read by the computer to perform the procedures described herein. Embodiments of the system may also be considered to be implemented as a non-transitory computer-readable storage medium, configured with a computer program, where the storage medium so configured causes a computer to operate in a specific and predefined manner to perform the functions described herein.

Furthermore, the system, processes and methods of the described embodiments are capable of being distributed in a computer program product comprising a computer readable medium that bears computer usable instructions for one or more processors. The medium may be provided in various forms, including one or more diskettes, compact disks, tapes, chips, wireline transmissions, satellite transmissions, internet transmission or downloadings, magnetic and electronic storage media, digital and analog signals, and the like. The computer useable instructions may also be in various forms, including compiled and non-compiled code.

Various embodiments have been described herein by way of example only. Various modification and variations may be made to these example embodiments without departing from the spirit and scope of the invention, which is limited only by the appended claims.

We claim:

1. A system for monitoring air in an indoor space, the system comprising:
at least one sensor located in the indoor space to generate monitoring data, the at least one sensor comprising at least one of a carbon dioxide sensor to detect carbon dioxide concentration within the indoor space, or a particle sensor to detect airborne particles within the indoor space;
a communication interface; and
at least one processor connected to the communication interface and in communication with the at least one sensor, the at least one processor being operable to:
receive, from the at least one sensor, monitoring data for an extended time period, the extended time period comprising at least one period when the indoor space is occupied and at least one period when the indoor space is unoccupied;
identify at least one region of interest in the monitoring data; and
determine an air change rate for the indoor space based on the at least one region of interest.

2. The system of claim 1, wherein each region of interest identified in the monitoring data comprises a portion of the monitoring data having a substantially constant rate of decay immediately followed by a portion of the monitoring data being substantially constant.

3. The system of claim 1, wherein, for each region of interest, the monitoring data at the start of the region of interest is greater than a pre-determined minimum value.

4. The system of claim 1, wherein, for each region of interest, the monitoring data at the end of the region of interest is greater than a pre-determined maximum value.

5. The system of claim 1, wherein:
the at least one sensor further comprises a barometric pressure sensor to detect pressure changes within the indoor space; and
the at least one processor is operable to:
identify transitional ventilation events in the monitoring data based on the pressure changes detected by the barometric pressure sensor; and
identify the at least one region of interest in the monitoring data based on the transitional ventilation events.

6. The system of claim 1, wherein the at least one processor is further operable to:
receive, from the at least one sensor, real-time monitoring data;
determine at least one of an occupancy in the indoor space based on the real-time monitoring data, or an airborne infection risk in the indoor space based on the real-time monitoring data; and
cause, via the communication interface, the occupancy or the airborne infection risk to be displayed.

7. The system of claim 6, wherein the at least one processor is operable to:
determine a safe occupancy capacity based on the air change rate; and
determine the airborne infection risk based on the safe occupancy capacity and the real-time monitoring data.

8. The system of claim 6, wherein:
the at least one sensor further comprises a microphone to detect acoustic volume levels within the indoor space; and
the at least one processor being operable to determine the occupancy or the airborne infection risk in the indoor space is further based on the acoustic levels detected by the microphone.

9. The system of claim 6, wherein:
the at least one sensor further comprises an ambient light sensor to detect indoor lighting in the indoor space; and
the at least one processor being operable to determine the occupancy is further based on the indoor lighting detected by the ambient light sensor.

10. The system of claim 6, wherein the at least one processor is further operable to:
compare the occupancy or the airborne infection risk to a respective pre-determined threshold value; and
in response to determining that the occupancy or the airborne infection risk exceeds the respective pre-determined threshold value, generate a notification to be provided at a user interface.

11. A method for monitoring air in an indoor space, the method comprising:
monitoring at least one of a carbon dioxide concentration or airborne particles in the indoor space for an extended time period to generate monitoring data, the extended time period comprising at least one period when the indoor space is occupied and at least one period when the indoor space is unoccupied; and
operating at least one processor to:
receive the monitoring data;
identify at least one region of interest in the monitoring data; and
determine an air change rate for the indoor space based on the at least one region of interest.

12. The method of claim 11, wherein each region of interest identified in the monitoring data comprises a portion of the monitoring data having a substantially constant rate of decay immediately followed by a portion of the monitoring data being substantially constant.

13. The method of claim 11, wherein, for each region of interest, the monitoring data at the start of the region of interest is greater than a pre-determined minimum value.

14. The method claim 11, wherein, for each region of interest, the monitoring data at the end of the region of interest is greater than a pre-determined maximum value.

15. The method claim 11, further comprising:
detecting pressure changes within the indoor space; and
operating the at least one processor to:
identify transitional ventilation events in the monitoring data based on the pressure changes detected; and
identify the at least one region of interest in the monitoring data based on the transitional ventilation events.

16. The method of claim 11, further comprising:
monitoring at least one of a carbon dioxide concentration or airborne particles in the indoor space in real-time to generate real-time monitoring data; and
operating the at least one processor to:
receive the real-time monitoring data;
determine at least one of an occupancy in the indoor space based on the real-time monitoring data, or an airborne infection risk in the indoor space based on the real-time monitoring data; and
cause the occupancy or the airborne infection risk to be displayed at a user interface.

17. The method of claim 16, comprising operating the at least one processor to:
determine a safe occupancy capacity based on the air change rate; and
determine the airborne infection risk based on the safe occupancy capacity and the real-time monitoring data.

18. The method of claim 16, further comprising detecting acoustic volume levels within the indoor space, wherein operating the at least one processor to determine the occupancy or the airborne infection risk in the indoor space is further based on the acoustic levels detected.

19. The method of claim 16, further comprising detecting indoor lighting in the indoor space, wherein operating the at least one processor to determine the occupancy is further based on the indoor lighting detected.

20. The method of claim 16, further comprising operating the at least one processor to:
compare the occupancy or the airborne infection risk to a pre-determined threshold value; and
in response to determining that the occupancy or the airborne infection risk exceeds the pre-determined threshold value, generate a notification to be provided at a user interface.
